(19) United States
(12) Patent Application Publication
(10) Pub. No.: US 2010/0215781 A1
(43) Pub. Date: Aug. 26, 2010

(54) THERAPEUTIC COMPOSITION COMPRISING OMEGA-3 POLYUNSATURATED FATTY ACID OR DERIVATIVE THEREOF, RESVERATROL AND GREEN TEA

(76) Inventor: Joar Opheim, Aptos, CA (US)

Correspondence Address: JONES DAY (for Nordic Naturals), 222 EAST 41ST. STREET, NEW YORK, NY, 10017-6702, US

(21) Appl. No.: 12/392,830

(22) Filed: Feb. 25, 2009

(51) Int. Cl. A61K 36/82

(52) U.S. Cl. 424/729

(57) ABSTRACT

A therapeutic composition is provided comprising: (1) at least one omega-3 polyunsaturated fatty acid, at least one pharmaceutically acceptable omega-3 polyunsaturated fatty acid derivative or mixtures thereof; (2) resveratrol or a resveratrol derivative; and (3) green tea or a green tea derivative, each in an amount, individually and in combination, effective for a therapeutic, preventive or nutritional activity in a subject in need thereof.

### BACKGROUND OF THE INVENTION

The present invention relates to therapeutic compositions suitable for supporting antiaging in a subject.

### SUMMARY OF THE INVENTION

The present invention provides a therapeutic composition comprising (1) at least one omega-3 polyunsaturated fatty acid, at least one pharmaceutically acceptable omega-3 polyunsaturated fatty acid derivative or mixtures thereof, (2) resveratrol or a resveratrol derivative, and (3) green tea or a green tea derivative, each in an amount, individually and in combination, effective for a therapeutic, preventive or nutritional activity in a subject in need thereof.

Also provided by the present invention is a method for supporting antiaging in a subject comprising periodically administering to the subject an effective amount of a composition (1) at least one omega-3 polyunsaturated fatty acid, at least one pharmaceutically acceptable omega-3 polyunsaturated fatty acid derivative or mixtures thereof, (2) resveratrol or a resveratrol derivative, and (3) green tea or a green tea derivative, each in an amount, individually and in combination, effective for promoting antiaging in the subject. As used herein, the term “administering” includes both the case where a third party administers the composition to the subject, and the case where the subject self-administers the composition.

### DETAILED DESCRIPTION OF EMBODIMENTS

### Omega-3 Polyunsaturated Fatty Acids

As used herein, the term “omega-3 polyunsaturated fatty acid(s)” refers to a family of unsaturated fatty carboxylic acids that have in common a carbon-carbon bond in the n-3 position (i.e., the third bond from the methyl end of the molecule). Typically, they contain from about 16 to about 24 carbon atoms and from three to six carbon-carbon double bonds. Omega-3 polyunsaturated fatty acids can be found in nature, and these natural omega-3 polyunsaturated fatty acids frequently have all of their carbon-carbon double bonds in the cis-configuration.

Examples of omega-3 polyunsaturated fatty acids include, but are not limited to, 7,10,13-hexadecatrienoic acid (sometimes abbreviated as 16:3 (n-3)); 9,12,15-octadecatetrienoic acid (α-linolenic acid (ALA), 18:3 (n-3)); 6,9,12,15-octadecatetraenoic acid (stearidonic acid (STD), 18:4 (n-3)); 11,14,17-eicosatrienoic acid (eicosatrienoic acid (ETE), 20:3 (n-3)); 8,11,14,17-eicosatetraenoic acid (eicosatetraenoic acid (ETA), 20:4 (n-3)); 5,8,11,14,17-eicosapentaenoic acid (eicosapentaenoic acid (EPA), (20:5 (n-3)); 7,10,13,16,19-docosapentaenoic acid (docosapentaenoic acid (DPA), 22:5 (n-3)); 4,7,10,13,16,19-docosahexaenoic acid (docosahexaenoic acid (DHA), 22:6 (n-3)); 9,12,15,18,21-tetracosapentaenoic acid (tetracosapentaenoic acid, 24:5 (n-3)); and 6,9,12,15,18,21-tetracosahexaenoic acid (tetracosahexaenoic acid, 24:6 (n-3)).

Eicosapentaenoic acid (EPA) and docosahexaenoic acid (DHA) are found in nature in fish oils, and have been used in a variety of dietary/therapeutic compositions. EPA and DHA are preferred omega-3 polyunsaturated fatty acids in the present invention.

### Omega-3 Polyunsaturated Fatty Acid Derivatives

As used herein, the term “omega-3 polyunsaturated fatty acid derivative(s)” refers to omega-3 polyunsaturated fatty acids that have been reacted with another compound or otherwise modified so that the omega-3 polyunsaturated fatty acid no longer contains a free carboxylic acid. Examples of omega-3 polyunsaturated fatty acid derivatives include salts, esters (such as ethyl esters) and glycerides of omega-3 polyunsaturated fatty acids.

As used herein, the term “glyceride” means a glycerol molecule (i.e., OHCH₂CHOHCH₂OH) in which one, two or all three of the hydroxyls have been esterified with a carboxylic acid, e.g., an omega-3 polyunsaturated fatty acid. Thus, “triglyceride” refers to glycerides in which all three hydroxyls on the glycerol have been esterified with (the same or different) carboxylic acids. “Diglyceride” refers to glycerides in which only two of the hydroxyls on the glycerol have been esterified with (the same or different) carboxylic acids. “Monoglyceride” refers to glycerides in which only one hydroxyl on the glycerol has been esterified with a carboxylic acid.

As used herein, the term “pharmaceutically acceptable” means that the material to which it refers is not harmful to the subject.

In one embodiment, the composition of the invention employs a mixture of omega-3 polyunsaturated fatty acids and/or derivatives that contain glycerides. For example, in one embodiment, the mixture contains about 35 wt. % triglycerides of EPA and about 25 wt. % triglycerides of DHA. In another embodiment the mixture can contain at least about 60 wt. % of a combination of EPA and DHA in a weight ratio of EPA:DHA of from about 1.4:1 to about 5:1, wherein the combination is at least about 60% (e.g., at least about 80% or at least about 90%) in the triglyceride form of the fatty acids and the balance is at least about 80% mono- and di-glycerides.

### Resveratrol

Resveratrol is a phytoalexin produced naturally by several plants (for example in the skin of red grapes) when under attack by pathogens such as bacteria or fungi. Resveratrol can also be made by chemical synthesis. Chemically, it is the polyphenol 3,5,4′-trihydroxystilbene. It can be in either the cis- or trans-configuration (or as mixtures of the two), with the trans-configuration being preferred for therapeutic/nutritional uses.

### Green Tea

As used herein, the term “green tea” refers to leaves obtained from the genus Camellia including C. sinensis and C. assaimica, or their hybrids, as well as products, such as extracts, obtained from such leaves. For instance, “green tea” includes freshly gathered green tea leaves, fresh green tea leaves that are dried immediately after gathering, fresh green tea leaves that have been heat treated before drying to inactivate any enzymes present, unfermented tea, instant green tea, and aqueous or oil-soluble extracts of these leaves.

Green tea extracts can be obtained from commercial sources (e.g., Nature's Resource, Mission Hills, Calif., or Herbasin, Beijing, China) or can be obtained directly from green tea leaves using extraction methods disclosed in European Patent No. EP 1402869 to Schneider, which is hereby incorporated by reference in its entirety. Green tea and green tea extracts can contain varying amounts of catechins, (−) epigallocatechin gallate (EGCG) and caffeine.

The compositions of the present invention contain (1) at least one omega-3 polyunsaturated fatty acid, at least one pharmaceutically acceptable omega-3 polyunsaturated fatty acid derivative or mixtures thereof, (2) resveratrol or a resveratrol derivative, and (3) green tea or a green tea derivative, each in an amount, individually and in combination, effective for a therapeutic, preventive or nutritional activity in a subject in need thereof. By “amount individually and in combination effective” is meant that each individual component is present in an amount sufficient to perform its function as well as the overall composition being in an amount sufficient to perform its overall function.

The compositions of this invention can contain other ingredients besides the three recited above. These include, but are not limited to, flavor agents, fillers, surfactants (e.g., polysorbate 80 and sodium lauryl sulfate), color agents including, e.g., dyes and pigments, sweeteners, antioxidants and additional ingredients.

### Flavor Agents

Useful flavor agents include natural and synthetic flavoring sources including, but not limited to, volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, banana, grape, berry, strawberry, raspberry, blueberry, blackberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof, chocolate, cocoa, almond, cashew, macadamia nut, coconut, mint, chili pepper, pepper, cinnamon, vanilla, tooty fruity, mango and green tea. Mixtures of two or more flavor agents may also be employed. When a flavor agent is used, the amount employed will depend upon the particular flavor agent used. However, in general, the flavor agent can constitute from about 5% to about 50% by weight of the composition.

### Color Agents

Useful color agents include, e.g., food, drug and cosmetic (FD&C) colors including, e.g., dyes, lakes, and certain natural and derived colorants. Useful lakes include dyes absorbed on aluminum hydroxide and other suitable carriers. Mixtures of color agents may also be employed. When a color agent is employed, the amount used will depend upon the particular color agent used. However, in general, the color agent can constitute from about 0.5% to about 5% by weight of the composition.

### Sweetening Agent

Natural and/or artificial sweetening agents can also be added to the composition. Examples of sweeteners include sugars such as sucrose, glucose, invert sugar, fructose, and mixtures thereof, saccharin and its various salts (e.g., sodium and calcium salt of saccharin), cyclamic acid and its various salts, dipeptide sweeteners (e.g., aspartame), dihydrochalcone, and sugar alcohols including, e.g., sorbitol, sorbitol syrup, mannitol and xylitol, and combinations thereof. Natural sweeteners that can be employed include, but are not limited to, luo han, stevia or mixtures thereof. Luo han sweetener is derived from luo han guo fruit (siraitia grosvenorii) that is mainly found in China. It is about 300 times sweeter by weight than sucrose. Luo han is commercially available from, e.g., Barrington Nutritionals (Harrison, N.Y.). Stevia is derived from a South American herb, Stevia rebaudiana. It can be up to about 300 times sweeter than sucrose. Because luo han and stevia have such a sweet taste, only a small amount need be used in the composition. When a sweetening agent is employed the amount used will depend upon the particular sweetening agent used. However, in general, the sweetening agent can constitute from about 0.0005% to about 30%, by weight of the composition. When a sweetener having a very sweet taste, such as luo han or stevia, is used, small amounts such as about 0.0005% to about 0.1% (for example about 0.005% to about 0.015% or about 0.002% to about 0.003%) by weight can be used.

### Additional Ingredients

The compositions of the present invention can contain additional ingredients. Examples of such additional ingredients include, but are not limited to, vitamins, minerals and/or herbs.

As used herein, the term “vitamin” refers to trace organic substances that are required in the diet. For the purposes of the present invention, the term vitamin(s) include, without limitation, thiamin, riboflavin, nicotinic acid, pantothenic acid, pyridoxine, biotin, folic acid, vitamin B12, lipoic acid, ascorbic acid, vitamin A, vitamin D, vitamin E and vitamin K. Also included within the term vitamin are the coenzymes thereof. Coenzymes are specific chemical forms of vitamins. Coenzymes include thiamine pyrophosphates (TPP), flavin mononucleotide (FMM), flavin adenine dinucleotive (FAD), Nicotinamide adenine dinucleotide (AND), Nicotinamide adenine dinucleotide phosphate (NADP), Coenzyme A (CoA), Coenzyme Q10 (CoQ10), pyridoxal phosphate, biocytin, tetrahydrofolic acid, coenzyme B12, lipoyllysine, 11-cis-retinal, and 1,25 -dihydroxycholecalciferol. The term vitamin(s) also includes choline, camitine, and alpha, beta, and gamma carotenes.

As used herein, the term “mineral” refers to inorganic substances, metals, and the like required in the human diet. Thus, the term “mineral” as used herein includes, without limitation, calcium, iron, zinc, selenium, copper, iodine, magnesium, phosphorus, chromium and the like, and mixtures thereof. Compounds containing these elements are also included in the term “mineral.”

As used herein, the term “herb” refers to organic substances defined as any of various often aromatic plants used especially in medicine or as seasoning. Thus, the term “herb” as used herein includes, but is not limited to, black currant, ginsing, ginko bilboa, cinnamon, and the like, and mixtures thereof. Compounds containing these elements are also included in the term “herb.”

Other ingredients that can be used include antioxidants, glucosamine and mixtures thereof.

The compositions of this invention are suitable for therapeutic and/or nutritional purposes in treating a subject in need of such treatment. As used herein, the term “subject” includes, but is not limited to, a non-human animal, such as a cow, monkey, horse, sheep, pig, chicken, turkey, quail, cat, dog, mouse, rat, rabbit, or guinea pig; and a human.

The amount of the composition of the invention that is effective will vary depending upon the condition being treated, and can be determined by standard clinical techniques. In addition, in vitro or in vivo assays can optionally be employed to help identify optimal dosage ranges. The precise dose to be employed will also depend on the relative amounts of the components of the compositions of the invention, route of administration, and the seriousness of the condition being treated and should be decided according to the judgment of the practitioner and each subject's circumstances. However, suitable effective dosage amounts for the compositions of the invention typically are at least about 2 grams per day, typically administered in the form of capsules containing at least about 1 gram of the composition per capsule. A typical dose should contain sufficient omega-3 acids or derivatives thereof to provide at least about 500 mg of a combination of EPA and DHA (or derivatives thereot). The dose should also contain about 1 mg to about 1500 mg or more of resveratrol, for example 25-500 mg, 50-300 mg or 100-120 mg per dose. The amount of green tea per dose can range from about 50 mg to about 1500 mg or more. The amount of green tea should provide about 50 to about 1500 mg (e.g., 50-500 mg) or more of total polyphenols. Of the total polyphenols, a minimum of about 45% should be EGCG. A typical dose can contain up to about 1200 mg EGCG.

The form in which the composition of the invention is administered to the subject is not critical. Typically, the composition is administered as a liquid or in a capsule.

The compositions of this invention can be used to promote antiaging (longevity) in a subject. The compositions can also help prevent damage to DNA and/or repair damage already done to DNA and/or diminish free radical damage.

The following examples demonstrate, but do not limit, the present invention.

### EXAMPLES

There are numerous variations on the embodiments of the present invention illustrated in the Examples which are possible in light of the teachings supporting the present invention. It is therefore understood that within the scope of the following claims, the invention may be practiced otherwise than as specifically described or exemplified.

### Example 1

The following composition represents one embodiment of the present invention:

| | | | Active | | |
|---|---|---|---|---|---|
| | | | Ingredient - | | |
| Parent | mg per | Percent | Percent of | mg per | Percent |
| Ingredient | 1 g cap | by weight | Parent | 1 g cap | by weight |
| Marine Lipid | 700 | 60.48% | | | |
| | | | EPA | 227.5 | 19.66% |
| | | | (Triglyceride | | |
| | | | form) - 35% | | |
| | | | DHA | 157.5 | 13.61% |
| | | | (Triglyceride | | |
| | | | form) - 25% | | |
| Polygonum | 120 | 10.37% | | | |
| Cuspidatum | | | | | |
| | | | Resveratrol - | 60 | 5.18% |
| | | | 50% | | |
| Green Tea | 250 | 21.60% | | | |
| Extract | | | Total | 245 | 2.20% |
| | | | polyphenols - | | |
| | | | 98% | | |
| | | | % Catechins | 187.5 | 16.20% |
| | | | found in | | |
| | | | polyphenols - | | |
| | | | 75% | | |
| | | | % EGCG | 112.5 | 9.72% |
| | | | found in | | |
| | | | catechins - | | |
| | | | 45% | | |
| Vitamin E | 15 | 1.30% | | | |
| Rosemary | 2.45 | 0.21% | | | |
| Extract | | | | | |
| Beeswax | 50 | 4.32% | | | |
| Lecithin | 20 | 1.72% | | | |

### Example 2

The following table shows the analysis of green tea from several sources.

| | | | % EGCG | % Catechins | |
|---|---|---|---|---|---|
| Green Tea | | | found in | found in | Total |
| No. | Form | Caffeine | catechins | polyphenols | Polyphenols |
| 1 | Powder | 2.00% | 40% | 70% | 90% |
| 2 | Powder | — | — | — | 40% |
| 3 | Powder | 2.00% | 51.09% | 85.40% | 99.11% |
| 4 | Powder | 0.42% | 45% | 75% | 98% |
| 5 | Powder | 8.95% | 11.90% | 41.64% | 50.71% |
| 6 | Powder | 0.05% | 46.21% | 75.69% | 96.07% |
| 7 | | | | | |
| 35% | Powder | ≦11% | ≧3% | — | ≧35% |
| 45% | Powder | — | ≧7% | ≧30% | ≧45% |
| 50% | Powder | ≦10% | ≧20% | ≧35% | ≧50% |
| 90% | Powder | — | ≧40% | ≧80% | ≧90% |
| 8 | | | | | |
| 30% | Powder | — | — | — | 30.00% |
| 98% | Powder | — | 45.00% | 75.00% | 98.00% |
| 9 | | | | | |
| 50% | Powder | 2.00% | 50.00% | — | 98.00% |
| 98% | Powder | — | — | 75.00% | 98.00% |
| Decaf | Powder | ≧2% | — | — | — |
| 10 | | | | | |
| 50% | Powder | 5.11% | 45.73% | 78.86% | 96.31% |
| 95% | Powder | 7.56% | 15.33% | 33.74% | 5.20% |
| 11 | | | | | |
| 60% | Powder | 5.90% | 28.40% | 41.10% | 68.20% |
| 60% | Powder | 0.44% | 23.45% | — | 62.64% |
| (decaf) | | | | | |
| 80% | Powder | 4.26% | 48.35% | 73.30% | 84.66% |
| 80% | Powder | 0.48% | — | 60.04% | 80.20% |
| (decaf) | | | | | |

Although specific embodiments of the present invention have been described, it will be understood by those of skill in the art that there are other embodiments that are equivalent to the described embodiments. Accordingly it is to be understood that the invention is not to be limited by the specific illustrated embodiments.

What is claimed is:

1. A therapeutic composition comprising: (1) at least one omega-3 polyunsaturated fatty acid, at least one pharmaceutically acceptable omega-3 polyunsaturated fatty acid derivative or mixtures thereof; (2) resveratrol or a resveratrol derivative; and (3) green tea or a green tea derivative, each in an amount, individually and in combination, effective for a therapeutic, preventive or nutritional activity in a subject in need thereof.

2. The therapeutic composition of claim 1 wherein component (1) comprises EPA, derivatives of EPA, DHA, derivatives of DHA or mixtures thereof.

3. The therapeutic composition of claim 2 wherein component (1) is a mixture comprising about 35 wt. % triglycerides of EPA and about 25 wt. % triglycerides of DHA.

4. The therapeutic composition of claim 2 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the fatty acids and the balance is at least about 80% mono and di-glycerides.

5. The therapeutic composition of claim 4 wherein the combination is at least about 80% in the triglyceride form.

6. The therapeutic composition of claim 4 wherein the combination is at least about 90% in the triglyceride form.

7. A method for supporting antiaging in a subject comprising periodically administering to the subject an effective amount of a composition (1) at least one omega-3 polyunsaturated fatty acid, at least one pharmaceutically acceptable omega-3 polyunsaturated fatty acid derivative or mixtures thereof, (2) resveratrol or a resveratrol derivative, and (3) green tea or a green tea derivative, each in an amount, individually and in combination, effective for promoting antiaging in the subject.

8. The method of claim 7 wherein component (1) comprises EPA, derivatives of EPA, DHA, derivatives of DHA or mixtures thereof.

9. The method of claim 8 wherein component (1) is a mixture comprising about 35 wt. % triglycerides of EPA and about 25 wt. % triglycerides of DHA.

10. The method of claim 8 wherein component (1) is a mixture comprising at least about 60 wt. % of a combination of EPA and DHA in a weight ratio of EPA:DHA of from about 1.4:1 to about 5:1, wherein the combination is at least about 60% in the triglyceride form of the fatty acids and the balance is at least about 80% mono and di-glycerides.

11. The method of claim 10 wherein the combination is at least about 80% in the triglyceride form.

12. The method of claim 10 wherein the combination is at least about 90% in the triglyceride form.
